(19) United States
(12) Patent Application Publication
(10) Pub. No.: US 2026/0287745 A1
(43) Pub. Date: Sep. 24, 2026

(54) VOLUME IMAGING SYSTEM WITH DIFFERENT BIAS PATTERNS

(71) Applicant: Method AI, Inc., Cleveland, OH (US)

(72) Inventor: Jonathan Fincke, Belmont, MA (US)

(73) Assignee: Method AI, Inc., Cleveland, OH (US)

(21) Appl. No.: 19/477,080

(22) PCT Filed: Apr. 19, 2024
(86) PCT No.: PCT/US2024/025582

§ 371 Date: Oct. 20, 2025

Related U.S. Application Data
(60) Provisional application No. 63/461,189, filed on Apr. 21, 2023.

(51) Int. Cl. G01S 15/89

(52) U.S. Cl. CPC G01S 15/8925; G01S 15/8927; G01S 15/8977; G01S 15/8993

(57) ABSTRACT

An ultrasound imaging system may include an array of ultrasonic transducers and a controller configured to selectively activate different portions of the array to determine different two-dimensional images. Each of the two-dimensional images is based upon signals from a different first portion of the array. Each of the two-dimensional images is based on element data signals from the ultrasonic transducers of the first portion of the array in response to application of a first electrical bias pattern applied to the first and second portions during a transmitting phase and a second electrical bias pattern applied to the first and second portions of the array during the receiving phase. The second electrical bias pattern includes a non-zero electrical bias applied to at least one ultrasonic transducer of the second portion. A volumetric image may be generated based on the different two-dimensional images.

### BACKGROUND

Ultrasound imaging technology uses ultrasound transducers for imaging in the medical field and for many other applications. Image quality, resolution, imaging depth as well as 3D and volume imaging may be essential performance requirements for particular applications. However, complexity, cost, size and shape of the ultrasound transducers are also important factors that may limit a given ultrasound imaging technology from being useful.

It is common to employ mechanically scanned 1D arrays, 2D ultrasound arrays connected to application specific integrated circuits (ASICs), and 2D row column arrays to realize a 3D and/or volume ultrasound imaging systems. The arrays are typically made from piezoelectric materials, capacitive micromachined ultrasound transducer (CMUT) elements, piezoelectric micromachined ultrasound transducer (PMUT) elements and optoacoustic source and receiver elements. More recently there have been efforts to develop row column arrays using electrostricter arrays to form volume images.

These common 3D ultrasound imaging techniques have many drawbacks; mechanical motion-based systems are hard to mass produce, have low volume imaging rates and can suffer from reliability issues. ASIC based systems are difficult to fabricate, expensive to develop, can have overheating issues which can be further exacerbated at higher frequencies. 2D arrays of CMUT and PMUT elements with integrated ASICs reduce many of the cost and fabrication challenges seen with conventional 2D arrays (PZT based) with ASICs by allowing the entire fabrication of the arrays and ASIC to be done in one chip microfabrication process, however, the imaging and reliability performance of the CMUT and PMUT elements has yet to match that of conventional PZT arrays. This performance gap has prevented strong adoption of CMUT and PMUT arrays in practice. Row column arrays (RCA's) using typical piezo ceramic overcome the issues of mechanical motion and ASIC based 3D/volume imaging systems, but they have a limited field of view that is restricted to imaging directly in front of the active aperture. Adding lenses to these RCAs can make the field of view larger but image quality and penetration depth can suffer. Further, the 90-degree field of views (FOV's) achieved with ASICs or mechanical systems is unlikely to be matched by RCAs with lenses. RCAs can also be fabricated with CMUT and or PMUT elements, however the performance gap relative to PZT materials remains for RCA array architectures as well.

The shortcomings of these common 3D and volume imaging approaches can be addressed by fabricating RCAs from materials or elements that can have their acoustic transducing properties varied by an external action (e.g. EM field, voltage, current etc.). Such examples of these materials or elements are CMUT and PMUT cells and electrostrictive (i.e. relaxor) materials. All three of these example element types have transducing properties that are proportional to the voltage applied to the element. This property of the elements enables unique transmit and receive beamforming approaches that can yield large FOV volumes with real time frame/volume rates without any mechanical motion. Previous works with CMUT arrays in particular have shown that volumes can be acquired with these RCA architectures. Despite the promising developments with CMUT and PMUT RCA's they still struggle to gain adoptions due to their performance shortcoming relative to standard array elements (PZT pillars).

Existing techniques for forming volumes with RCAs with electrostrictive elements require either data acquisition rates and 3D/volume image formation rates that are prohibitively slow for most clinical applications or the presence of dedicated electronic circuitry on or in very close proximity to the array to realize fast 3D/Volume imaging which prohibit miniaturization of theses arrays for many applications (e.g. surgical procedures, NDT applications in tight spaces etc.). This electronic circuitry primarily consists of switches or other components needed to ground elements where they are intended to have zero transducing capacity. In the case of electrostricter materials this would correspond to elements that have a zero-bias applied to them.

### BRIEF DESCRIPTION OF THE DRAWINGS

FIG. 1 is a diagram schematically illustrating portions of an example ultrasound imaging system capturing images of an internal organ.

FIG. 2 is a perspective view schematically illustrating portions of an example imaging device of the example system of FIG. 1, the example imaging device comprising an array of ultrasonic transducers.

FIG. 3 is a perspective view schematically illustrating a portion of the example imaging device of FIG. 2.

FIG. 4 is a diagram illustrating an example of how a portion or aperture of an array may be “walked” along array to use different portions of the array to form corresponding different two-dimensional images and process the data using synthetic aperture methods to improve image resolution along the “walking” direction.

FIG. 5 is a diagram comparing two alternative techniques for walking the aperture along the array.

FIG. 6 is a flow diagram illustrating an example method for generating a volumetric image based upon a combination of different two-dimensional images.

FIG. 7 is a diagram illustrating an example method wherein a first electrical bias pattern defining the aperture is applied during a transmit phase and a second electrical bias pattern is applied during a receiving phase, the second electrical bias pattern comprising a non-zero electrical bias which is timed or phased to steer or look away from the aperture.

FIG. 8 is a diagram illustrating an example method wherein a first electrical bias pattern defining the aperture is applied during a transmit phase, the first electrical bias pattern comprising a non-nonzero electrical bias is timed, phased or steered away from the aperture and wherein a second electrical bias pattern is applied during a receiving phase, the second electrical bias pattern comprising a non-nonzero electrical bias which is timed, phased or steered to look away from the aperture.

FIG. 9 is a diagram illustrating an example method wherein a first electrical bias pattern defining the aperture is applied during a transmit phase, the first electrical bias pattern comprising a non-zero electrical bias having a first polarity to regions outside the aperture and wherein a second electrical bias pattern is applied during receiving phase, the second electrical bias pattern comprising a non-zero bias having a second opposite polarity in the regions outside the aperture.

FIG. 10 is a diagram illustrating an example method wherein a first electrical bias pattern defining the aperture is applied during a transmit phase and a second electrical bias pattern is applied during a receiving phase, wherein a nonzero electrical bias is applied to the aperture and a zero electrical bias is applied to portions outside the aperture during the transmit phase.

FIG. 11 Is an example beam plot resulting from element data produced by the application of the first electrical bias pattern and the second electrical bias pattern of FIG. 10, FIG. 11 including a beam plot for a single image and a bam plot resulting from an additional application of a synthetic aperture.

FIG. 12 in the diagram illustrating an example method wherein electrical bias patterns are applied such that element data from transmit/receive phases cancel out or eliminate signal contributions from unused portions of the array (outside the aperture) to form each final 2D image, wherein such cancellation may occur at any of the various stages during processing of the element data and wherein the 2D images are used for forming a volume image.

FIG. 12A is a diagram illustrating an example method wherein multiple partial or sub-images from multiple transmit size receive operations are used to form a whole two-dimensional image, wherein multiple whole two-dimension images used to form a whole three-dimensional or volumetric image.

FIG. 13 is a diagram illustrating an example method wherein the electrical bias patterns applied to the array are applied during multiple transmit/receive operations to cancel or remove signal contributions from those transducers of the unused portions of the array.

FIG. 14 is a diagram illustrating an example method wherein electrical bias patterns are applied during multiple transmit/receive operations to cancel contributions from transducers outside an aperture during the transmit and receive phases.

FIG. 15 is a diagram illustrating beam plot resulting from element data produced by the method of FIG. 14.

FIG. 16 illustrates an example electrical bias pattern scheme that may be applied to reduce or eliminate parasitic capacitance and/or to reduce or limit acoustic signal contributions from unused portions of the array for a particular two-dimensional image.

FIG. 17 illustrates an example electrical bias pattern scheme that may be applied to reduce or eliminate parasitic capacitance and/or to reduce or limit acoustic signal contributions from unused portions of the array for a particular two-dimensional image.

FIG. 18 illustrates an example electrical bias pattern scheme that may be applied to reduce or eliminate parasitic capacitance and/or to reduce or limit acoustic signal contributions from unused portions of the array for a particular two-dimensional image.

FIG. 19 is a slice of an example volumetric ultrasound image from an example simulation in which the electrical bias pattern scheme of FIG. 14 is applied during transmit and receive phases for two-dimensional images that are used to form the volumetric image.

FIG. 20 is a diagram illustrating elevation, azimuth and depth of an example volume image.

FIG. 21 is an orthogonal image slice of the volumetric ultrasound image of FIG. 19.

FIG. 22 is a tri-plane image of the volumetric ultrasound image of FIG. 19.

FIG. 23 is a beam plot illustrating beam thickness in an elevation direction.

FIG. 24 is a beam plot illustrating beam thickness in an azimuth direction.

FIG. 25 is an ultrasound image illustrating an example slice of a volume generated pursuant to the method of FIG. 5, wherein a zero bias is applied to unused portions of an array when capturing element data for each two-dimensional image that form the volume image.

FIG. 26 is an ultrasound image illustrating an example slice of a volume generated pursuant to the method of FIG. 5, wherein a non-zero bias is applied to unused portions of an array when capturing element data for each two-dimensional image that form the volume image.

FIG. 27 as an ultrasound image illustrating various examples point to targets during an example simulation of an application of synthetic aperture processing.

FIG. 28 is an ultrasound image illustrating an example volume image performed using the method of FIG. 5, with the application of a non-zero bias to unused portions of the array for each two-dimensional image, without the application of synthetic aperture processing.

FIG. 29 is an ultrasound image illustrating an example volume image performed using the method of FIG. 5, with the application of a non-zero bias to unused portions of the array for each two-dimensional image, with the application of synthetic aperture processing.

FIG. 30 is a maximum projection rendering of the volume formed using a walking aperture with zero bias applied to the unused portions of the array, wherein the volume contains point targets and a spherical hyperechoic target.

FIG. 31 is a 2D image slice through the elevation axis from the volume showing point targets extending and the hyperechoic target.

FIG. 32 is a 2D image slice through the azimuthal axis showing the point targets and hyperechoic target.

FIG. 33 is a 2D image slice through the depth axis from the volume showing the hyperechoic target.

FIG. 34 is a beam plot along the azimuth axis from the volumetric image shown in FIGS. 30-33, wherein the beam plot shows the resolution of the imagery in the azimuth direction at various depths.

FIG. 35 is a beam plot along the elevation axis from the volumetric image shown in FIGS. 30-33, wherein the beam plot shows the resolution of the imagery in the elevation direction at various depths.

FIG. 36 is a maximum projection rendering of the volume formed using a walking aperture with non-zero bias applied to the unused portions of the array, wherein the volume contains point targets and two spherical hyperechoic targets.

FIG. 37 is a 2D image slice through the elevation axis from the volume in FIG. 36 which shows point targets and the hyperechoic spherical targets.

FIG. 38 is a 2D image slice through the azimuthal axis showing the point targets and hyperechoic targets.

FIG. 39 is a 2D image slice through the depth axis from the volume showing the hyperechoic target.

FIG. 40 is a beam plot along the azimuth axis from the volumetric image shown in FIGS. 36-39. The beam plot shows the resolution of the imagery in the azimuth direction at various depths.

FIG. 41 is a beam plot along the elevation axis from the volumetric image shown in FIGS. 36-39. The beam plot shows the resolution of the imagery in the elevation direction at various depths.

FIG. 42 is a 2D slice of a volume image formed with an ultrasound device using a walking aperture with zero bias applied to the unused portions of the array.

FIG. 43 is a 2D slice of a volume image formed with an ultrasound device using a walking aperture with non-zero bias applied to the unused portions of the array.

### DETAILED DESCRIPTION OF EXAMPLES

Disclosed are techniques for enabling a walking aperture on electrostrictive RCA's while keeping significant portions of the array active (a non-zero bias applied). The disclosed techniques may be employed in other ultrasound imaging systems as well, such as CMUT and PMUT systems. By walking an aperture with significant portions of the array active, the issue of parasitic capacitance is eliminated as well as the need for extra electrical components on the array which enables the miniaturization of these arrays that was not previously achievable. Further, this technique is not limited to a certain array geometry (e.g. curved vs. flat). When combining this technique with curved (in one, two or three dimensions), large FOV volumes can be captured with real time acquisition rates without mechanical motion, or ASICs partnered with fully connected 2D arrays.

Disclosed are example ultrasound imaging systems that form three-dimensional or volume images. The volume images are formed based upon a stack of different two-dimensional images calculated, generated from, derived from or determined based upon acoustic waves radiated from and sensed by an array of ultrasound transducers. Each two-dimensional image is based upon acoustic waves produced using the entirety of a portion of the array. The waves subsequently interact with the propagation medium which results in waves that return to the array. These waves are sensed on the array by activation of a different selected portion of the array. In some implementations, the different two-dimensional images may be the result of a particular array aperture (a selected portion of the array) being sequentially “walked” across the array of ultrasound transducers. Because the volume image is produced by walking the aperture, the ultrasound array may be fixed or stationary during such sensing. As result, mechanical movement or wobbling of the array may be avoided, potentially reducing the complexity, cost and size of the ultrasound imaging system.

The example volume imaging systems may comprise ultrasonic transducers which individually comprise electrically conductive layers sandwiching a dielectric layer therebetween. Such ultrasonic transducers may be individually activated by selectively applying a bias voltage to selected ultrasonic transducers. In some implementations, the ultrasonic transducers are in a row-column array, wherein the selective activation of individual transducers is achieved by selectively providing a signal voltage across selected rows/columns and by selectively providing a bias voltage across selected columns/rows. This configuration facilitates a wide imaging field-of-view (not restricted to dimensions of the array). In addition, this configuration may reduce the number of individual connections required for the array and may avoid the need for an application-specific integrated circuit (ASIC).

When forming one of the different two-dimensional images used to form the volume image, the example volume imaging systems apply a non-zero electrical bias (voltage) to those unused portions of the array, those portions of the array that are not intended to be utilized to form the particular two-dimensional image (i.e. any contribution of these portions to the imagery is undesired). The non-zero electrical bias reduces the inherent capacitance (parasitic capacitance) of the ultrasonic transducers of the unused portions. The application of the non-zero electrical bias to the unused portions of the array reduces the extent to which the unused portions of the array absorb energy during one or both transmission and reception phases of the ultrasound transducers of the portion of the array being used to form the two-dimensional image. This may result in enhanced resolution and quality of the two-dimensional images as well as enhanced resolution and quality of the volume image formed from the two-dimensional images. The enhanced resolution and quality of the two-dimensional images is driven by the increase in array sensitivity that comes from reducing the effect of parasitic capacitance.

For purposes of this application, the term “processing unit” shall mean a presently developed or future developed computing hardware that executes sequences of instructions contained in a non-transitory memory. Execution of the sequences of instructions causes the processing unit to perform steps such as generating control signals. The instructions may be loaded in a random-access memory (RAM) for execution by the processing unit from a read only memory (ROM), a mass storage device, or some other persistent storage. In other embodiments, hard wired circuitry may be used in place of or in combination with software instructions to implement the functions described. For example, controller 92 may be embodied as part of one or more application-specific integrated circuits (ASICs). Unless otherwise specifically noted, the controller is not limited to any specific combination of hardware circuitry and software, nor to any particular source for the instructions executed by the processing unit.

For purposes of this disclosure, unless otherwise explicitly set forth, the recitation of a “processor”, “processing unit” and “processing resource” in the specification, independent claims or dependent claims shall mean at least one processor or at least one processing unit. The at least one processor or processing unit may comprise multiple individual processors or processing units at a single location or distributed across multiple locations.

For purposes of this disclosure, the phrase “configured to” denotes an actual state of configuration that fundamentally ties the stated function/use to the physical characteristics of the feature proceeding the phrase “configured to.”

For purposes of this disclosure, unless explicitly recited to the contrary, the determination of something “based on” or “based upon” certain information or factors means that the determination is made as a result of or using at least such information or factors; it does not necessarily mean that the determination is made solely using such information or factors. For purposes of this disclosure, unless explicitly recited to the contrary, an action or response “based on” or “based upon” certain information or factors means that the action is in response to or as a result of such information or factors; it does not necessarily mean that the action results solely in response to such information or factors.

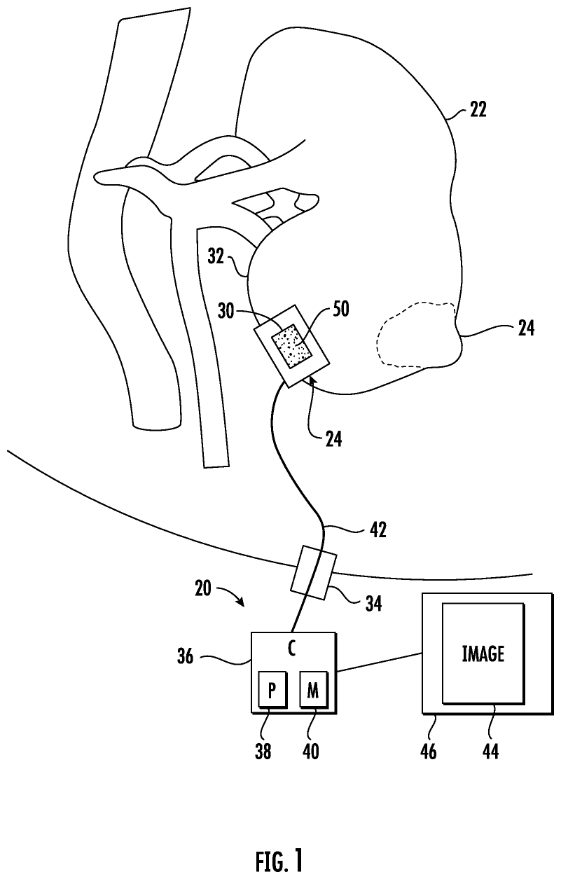

FIG. 1 is a perspective view illustrating portions of an example ultrasound imaging system 20 capturing images of an internal organ 22 (shown as a kidney). In the example illustrated, imaging system 20 captures the image of a tumor 24 on kidney 22 to facilitate medical diagnosis or treatment of the tumor 24. Imaging system 20 may be utilized to image other organs for other purposes. As shown by FIG. 1, imaging system 20 comprises an imaging device 30 attached to the surface 32 of the internal organ 22. In the example illustrated, imaging device 30 comprises an array 50 of ultrasound transducers. In the example illustrated, imaging device 30 is attached on a side of the internal organ 22 opposite to that of tumor 24 so as to not interfere with the manipulation of a surgical tool that may interact with tumor 24. In some implementations, imaging device 30 comprises an ultrasound transducer.

In some implementations, imaging device 30 may be contained within a housing 51 which is configured to be directly attached to the surface 32 of organ 22. In some imaging device 30 may be contained within housing that releasably connects or attaches to a carrier, such as a case, which attaches to the surface 32 of organ 22, wherein both the housing and/or the carrier are configured to be passed through a percutaneous access device 34, such as a trocar. The direct attachment of the housing or the direct attachment of the carrier to the surface 32 may be facilitated by passive vacuum, an active vacuum, grippers/hooks and/or a bio adhesive.

As further shown by FIG. 1, imaging system 20 may further comprise a controller 36 that comprises a processor or processing unit 38 which follows instructions stored or otherwise provided in a non-transitory computer-readable medium 40. Such instructions direct the processor 38 to carry out the imaging using imaging device 30. In such an implementation, controller 36 may communicate with the imaging device 30 across an electrical signal transmitting line or cable 42 that passes through access device 34 and is connected to imaging device 30.

In some implementations, controller 36 may comprise a beam former and an image processor. Functioning as a beam former, controller 36 may include a plurality of channels for generating transmit waveforms and/or receiving echoes or acoustic signals. Relative delays and/or apodization focus the transmit waveforms or received signals for forming beams. As will be described hereafter, the beam former provided by controller 36 may be connected to the top and bottom electrodes of the array of imaging device 30 to individually activate the ultrasound transducers of imaging device 30.

Functioning as an image processor and beamformer, controller 36 receives element data from the ultrasound transducers. Based upon the element data received from different portions of the array of ultrasound transducers, controller 36 may determine multiple different two-dimensional images. Element data refers to data that is based upon generated transmit waveforms and corresponding sensed or receiving acoustic echoes or signals, a transmit/receive pair or operation. Each of the two-dimensional images may be based upon a single set of element data resulting from a single transmit and receive phase from the beam former using a particular portion of the array of ultrasound transducers or may result from a combination of multiple sets of element data (or their corresponding intermediate images) resulting from multiple transmit and receive phases from the beam former using the particular portion of the array of ultrasound transducers. Controller 36 may combine the multiple different two-dimensional images to form the volume image 44.

Although the imaging device 30 is illustrated as being distinct form controller 36, as communicating with controller 36 through access device 34 and as being retained on an organ surface, in other implementations or with other uses, imaging device 30 may be located on an anatomy exterior when imaging or may be located inside an organ or tumor when imaging. In some implementations, communication with the imaging device 30 is performed in a wired fashion. In other implementations, communication may be performed wirelessly.

Although controller 36 is illustrated as being distinct from and connected to imaging device 30 and its array 50 by cable 42, in some implementations, controller 36, or functions of controller 36, may be provided by a processing unit and memory supported by imaging device 30 which is illustrated as being supported on a surface of organ 22. For example, the beam former may be provided as part of circuitry associated with the imaging device 30. In such implementations, rather than imaging device 30 transmitting analog signals through cable 42 to controller 36, the processing unit provided as part of the imaging device 30, may transmit digital signals, in a wired or wireless fashion, to display 46 or to an additional controller or processing unit that then causes display 46 to present image 44 based upon the received digital signals.

As indicated above, in some implementations, imaging device 30, along with the supported controller 36 or functions of controller 36, may be alternatively positioned within organ 22 or along a surface of the anatomy of the patient. In some implementations, imaging device 30 and controller 36 may be provided as part of a handheld imaging system that is to be positioned along the exterior anatomy of a patient to image interior portions of the anatomy. In some implementations, the controller 36 may communicate with the display 46 in a wireless fashion.

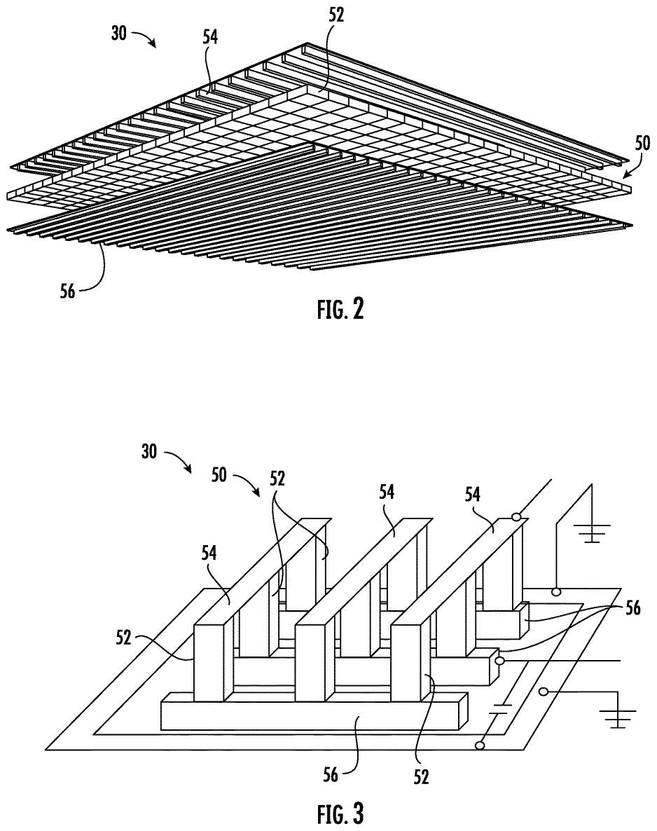

FIGS. 2 and 3 schematically illustrate one example of imaging device 30. Imaging device 30 comprises an array 50 of ultrasonic transducers 52 arranged in a row-column array. Each of transducers 52 comprises a pillar of a dielectric material, such as a Piezo-based material or crystal (e.g., a Piezo ceramic), wherein the pillar/transducer 52 may be activated through the creation of a voltage differential across the pillar.

In the example illustrated, each pillar has a first portion electrically connected to a top electrode 54, wherein top electrodes 54 are arranged in rows and are connected to corresponding rows of the transducers 52. Top electrodes 54 serve as a signal lines, transmitting an electrical signal with controlled time delayed signal having a controlled weight to each of the transducers 52 in the corresponding row.

Each of transducers 52 has a second portion electrically connected to a bottom electrode 56, wherein bottom electrodes 56 are arranged in columns and are connected to corresponding columns of transducers 52. Bottom electrodes 56 serve as bias lines, transmitting an electrical bias (zero or non-zero) to each of transducers 52 in the corresponding column.

Individual pillars/ultrasonic transducers 52 may be activated by transmitting both an electrical signal and an electrical bias voltage to the first and second portions of the pillar/ultrasonic transducer 52 during a transmit phase. Individual ultrasonic transducers 52 may be activated with the transmission of electrical bias voltage to the second portion of the pillar/ultrasonic transducer 52. Individual ultrasonic transducers 52 are in an inactive state when its bottom electrode 54 is at a zero electrical bias. In some implementations, an individual ultrasonic transducer 52 may be inactive in response to the lack of a voltage differential across the transducer 52.

In such a fashion, individual pillars/ultrasonic transducers 52 may be activated by controller 36 without the controller 36 having to be individually connected to each and every transducer 52. In the example illustrated, array 50 is part of an example electrostrictive transducer array. In other implementations, imaging device 30 may comprise an array of ultrasonic transducers in the form of a capacitive micro machine ultrasound transducer (CMUT) or a Piezo electric micro machine ultrasound transducer array (PMUT) array. Although illustrated as being substantially planar, in other implementations, array 50 may be arcuate or curved. Although the pillars forming transducers 54 are schematically illustrated as being arranged in orthogonal columns and rows, in other implementations, the pillars forming transducers 52 may have non-orthogonal layouts. For example, kerfs separating individual pillars 52 may be angled to form angled rows and/or angled columns of pillars 52, nonorthogonal to edges of the array 50.

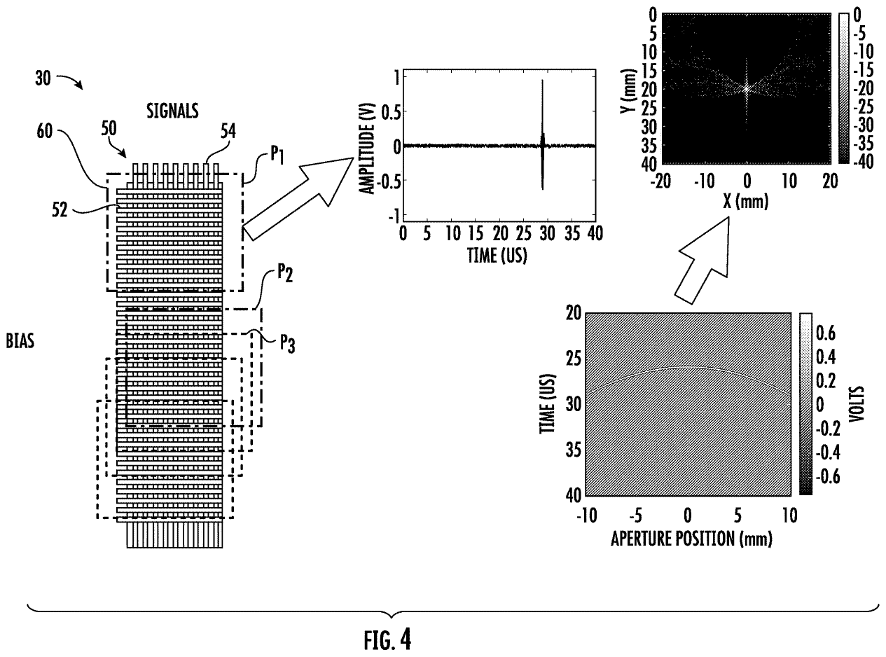

FIG. 4 illustrates an example of how a first portion or aperture 60 of the array 50 may be “walked” along the array 50 to use different portions of the array 50 to form corresponding different two-dimensional images. For example, FIG. 4 illustrates aperture 60 in a first position P1, a second position P2, a third position P3, and so along array 50. A different two-dimensional image may be determined at each of such positions. The resulting multiple two-dimensional images may be then combined to form a volume image.

Each aperture 60 contains those ultrasound transducers that are currently being used to form the particular two-dimensional image, a first portion of the ultrasound transducers of array 50 that are to be used for forming the particular two-dimensional image. Those ultrasound transducers 52 outside of aperture 60 are unused. This second portion 62 of array 50 includes those ultrasound transducers of array 50 that are not being used to form the particular two-dimensional image. The selective activation to define the current aperture 60 may be achieved with the selective application of bias voltages by bottom electrodes 56 to particular subsets of the ultrasound transducers of array 50.

In some implementations, the first portions or aperture 60 may be sequentially moved along the array 50 such that the different aperture 60 partially overlap adjacent prior or future aperture 60. In some implementations, the aperture 60 may be sequentially moved along the array 50 such that the different apertures 60 do not overlap, including exclusive sets of ultrasound transducers 52.

FIG. 5 illustrates two alternative techniques for walking aperture 60 along array 50. With a first technique 64, the beam former of controller 36 defines aperture 60 through the application of an electrical bias voltage to a selected first portion of the array 50. Beam former of controller 36 applies a zero electrical bias, a zero-bias voltage, to those ultrasound transducers not contained with aperture 60, the second portion 62 of array 50 not being used to form the current two-dimensional image.

With technique 64, portion 62 of array 50 has a structure of a capacitor, electrodes 54 and 56 sandwiching a dielectric material, transducer 52, therebetween. As result, with technique 64, array 50 experiences parasitic capacitance in portion 62. The capacitor by electrodes 54 and 56 and transducer 52 absorbs energy from the electrical signal being transmitted across the signal lines, top electrodes 54. During the transmission phase during which waveforms are generated, this parasitic capacitance reduces the strength of the transmitted waveforms, reducing the depth of the ultrasound scan. During the receiving phase during which echoes are sensed and converted to electrical pulses, this parasitic capacitance reduces the image quality by reducing the sensitivity of the array elements and the array overall which degrades the quality of the ultrasound scan. The process of walking the aperture consists of sequentially activating or utilizing portions of an array to acquire data and processing this data to form imagery which could be 2D or 3D imagery.

With a second technique 74, the beam former of controller 36 also defines aperture 60 through the application of electrical bias voltage to a selected first portion of the array 50. However, rather than applying a zero electrical bias, the beam former of controller 36 also applies a non-zero electrical bias (a “smart bias”) to at least one, a majority, over 90% or all of the ultrasound transducers in portion 62. The applied non-zero electrical bias prevents parasitic capacitance in portion 62, increasing image scanning depth and image quality.

The non-zero bias is applied by transmitting the non-zero electrical bias to each of the bottom electrodes 56 in region 62. The non-zero electrical bias is at a voltage such that parasitic capacitance is reduced or eliminated. The particular nonzero electrical bias voltage may depend on the thickness of the electrostrictive material and/or properties of the element architecture. In some implementations, the non-zero bias across the pillar of the transducer 52 (the piezo or ceramic material) may be greater than 1 kV/cm and no greater than 8 kV/cm (not much more than 7 kV/cm). For a 160 um thick piece of relaxor material (the pillar of transducer 52), a bias voltage range of approximately 10-140 V may be used, although the most likely range is 10-80 V.

In some implementations, the non-zero bias may be uniform across portion 62. In some implementations, the nonzero bias may vary across portion 62. For example, the non-zero electrical bias may be tapered or provided in some other pattern along portion 62.

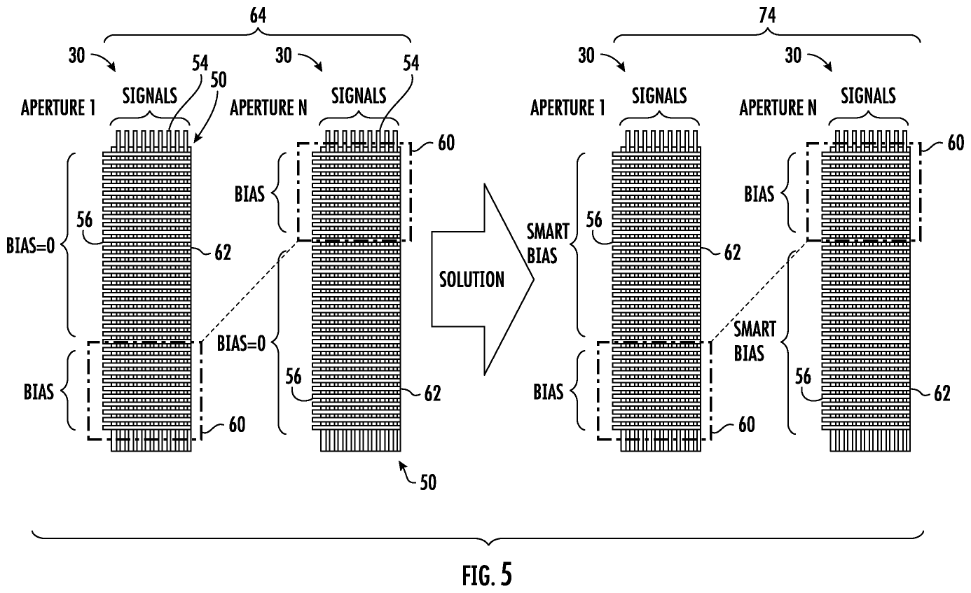

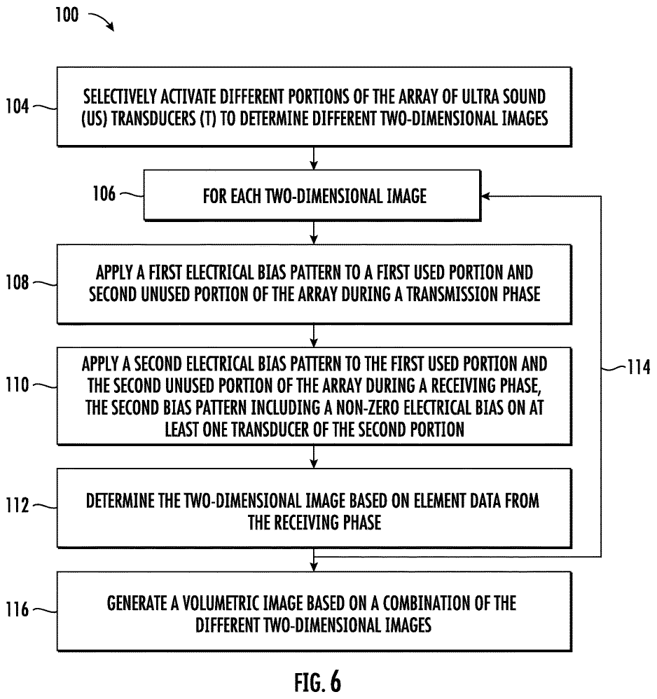

FIG. 6 is a flow diagram of an example method 100 that may be carried out by controller 36 with imaging device 30. Instructions contained in memory 40 may direct processor 38 to carry out method 100 to produce a volume image, such as a volume image of tumor 24 (shown in FIG. 1). As indicated by block 104, the beam former of controller 36 selectively activates different portions of array 50 of ultrasound transducers 52 to determine, calculate or form two-dimensional images.

As indicated by block 108, for each of the two-dimensional images that are determined in block 104, the beam former of controller 36 applies a first electrical bias pattern during a transmission or waveform producing phase to at least a portion of array 50 comprising a first used portion and a second unused portion of the array 50. The first electrical bias pattern may comprise a pattern of non-zero electrical bias voltages applied to both aperture 60, the first portion of array 50 to be used for forming the particular two-dimensional image, and to a second unused portion of array 50, the second portion array 50 not being used for forming the particular two-dimensional image. In some implementations, not all of the ultrasound transducers provided on array 50 are necessarily part of either aperture 60 or the second unused portion 62. As will be described hereafter, in some implementations, during the transmission interface, the beam former of controller 50 may apply a zero electrical bias to those ultrasound transducers of portion 62.

As indicated by block 110, for each of the two-dimensional images that are determined in block 104, the beam former of controller 36 applies a second electrical bias pattern during a receiving or echoes/signal sensing phase to at least a portion of array 50 comprising the first used portion and the second unused portion of the array 50. The second electrical bias pattern may comprise a pattern of non-zero electrical bias voltages applied to both aperture 60, the first portion of array 50 to be used for forming the particular two-dimensional image, and to the second unused portion 62 of array 50, the second portion 62 of array 50 not being used for forming the particular two-dimensional image. In some implementations, the second electrical bias pattern is different than the first electrical bias pattern. In some implementations, array 50 may comprise a third portion in addition to the first portion and the second portion which receive the first electrical bias pattern of non-zero electrical bias voltages. Said another way, in some implementations, the array 50 may comprise at least two portions, portions 60 and 62, with the possibility of additional portions which may or may not be at the same non-nonzero voltage as portion 62.

As indicated by block 112, the image processor of controller 50 determines the particular two-dimensional image based upon the raw or refined/processed element data which is based upon the acoustic echo sensed by the ultrasound transducers of aperture 60. The refinement/processing of the data may include filtering, down sampling, up sampling, shifting (phase or time delay). As will be described hereafter, the image processor of controller 50 applies one or more of various techniques for mitigating or eliminating any echo signals that may be produced by the activation of those ultrasound transducers in portion 62. The particular two-dimensional image for a particular location of aperture 60 (amongst the multiple different positions of aperture 60 during a walking of aperture 60 across array 50) may be based upon the single operation, a single set of raw or refined/processed element data produced during a single transmit and a single receive phase, of the beam former of controller 50.

In some implementations, the particular two-dimensional image for a particular location of aperture 60 may be based upon multiple sets of raw or refined/processed element data produced by multiple transmit and receive phases (multiple operations) provided by the beam former of controller 50. In such implementations, the particular two-dimensional image may be formed directly from the multiple sets of raw or refined/processed element data or may be indirectly formed from the multiple sets of raw or refined/processed element data. For example, each set of raw or refined/processed element data may be used to form a corresponding intermediate two-dimensional image, wherein the multiple intermediate two-dimensional images are combined (resulting in cancellation) to form the final two-dimensional image of block 104 that is to be used for forming the volume image. As indicated by arrow 114, blocks 108, 110 and 112 are repeated for each different location or position of aperture 60 along array 50, wherein a different two-dimensional images formed for each different location or position of aperture 60.

Because the beam former of controller 36 applies a non-zero electrical bias to at least one ultrasound transducer of the second portion during at least the receive phase, the phase during which ultrasound echoes are sensed, parasitic capacitance during the receive phase is reduced to improve ultrasound image scanning depth and image quality. As will be described hereafter, in some implementations, the non-zero bias voltage applied to those ultrasound transducers of aperture 60 during the transmit phase may be sufficiently strong to overcome any degradation caused by parasitic capacitance. This solution is generally not available during the receiving phase. In some implementations, the beam former of controller 36 may likewise apply a non-nonzero bias voltage to the ultrasound transducers of the unused portion 62 during the transmit phase to further reduce any impact from parasitic capacitance or to avoid having to apply a sufficiently strong transmit signal voltage to the ultrasound transducers of aperture 60 to overcome parasitic capacitance effects. The processes set forth in blocks 108, 110 and 112 are performed for each different portion of the array, for each different position of the aperture 60, and therefore, for each corresponding two-dimensional image being generated.

As indicated by block 116, the image processing component of controller 36 generates a volumetric or volume image based upon a combination of the different two-dimensional images as determined in block 104. This volume image may be stored and such or presented on display 46 (shown in FIG. 1). This volume image may guide a medical practitioner in the positioning of a surgical instrument when treating tumor 24. The enhanced ultrasound image depth and size or resolution may improve the ability of the medical practitioner to treat tumor 24 or to carry out medical diagnosis. The enhanced images may also be used for other applications such as non-destructive testing and therapeutic ultrasound.

Although the application of a non-zero bias to the transducers 52 in portion 62 mitigates or prevents parasitic capacitance, the application of the nonzero bias also activates such transducers 52 in portion 62 will cause transducer in 62 to radiate unwanted acoustic energy on transmit and produce voltages in response to acoustic waves that interact with the element on receive. The radiated acoustic energy and receive voltages from 62 will interfere with the acoustic signals from the transducers 52 in aperture 60. FIGS. 7-9 illustrate example methods for mitigating or eliminating such potential interference.

In the example shown in FIG. 7, the beam former of controller 36 applies a first electrical bias pattern 104 during the transmit (TX) phase. Pattern 104 defines aperture 60 (at a first particular position for a particular two-dimensional image) which contains transducers 52 that are to be used for forming the particular two-dimensional image and unused portion 62 which contains those transducers which are not to be used for forming the particular two-dimensional image. In the example illustrated, the beam former of controller 50 applies a non-nonzero electrical bias to those transducers in aperture 60, activating such transducers 52. The beam former of controller 36 applies a zero electrical bias or, in some other implementations, a non-nonzero bias that substantially reduces or eliminates the radiation of acoustic energy or contribution of signal during receive and a non-nonzero bias to those transducers of portion 62. During the transmit phase, the signal strength may be sufficiently high so as to overcome any energy losses due to parasitic capacitance.

During the receiving (RX) phase, the beam former of controller 36 applies a second electrical bias pattern 108. With electrical bias pattern 108, a non-zero electrical bias is applied to the transducers 52 of aperture 60. In addition, a nonzero electrical bias is applied to at least one, a majority of, at least 90%, or all of the transducers 52 of portion 62. As discussed above, this nonzero electrical bias activates the transducers 52 of portion 62 to prevent parasitic capacitance. In the example illustrated, non-zero electrical bias applied to the transducers 52 of portion 62 is timed or phased to steer or look away from aperture 60 as indicated by arrows 112. By steering (using bias voltages applied across the array elements) transducers 52 of portion 62 away from aperture 60, acoustic interference with the signals produced by the transducers 52 of aperture 60 is reduced or eliminated.

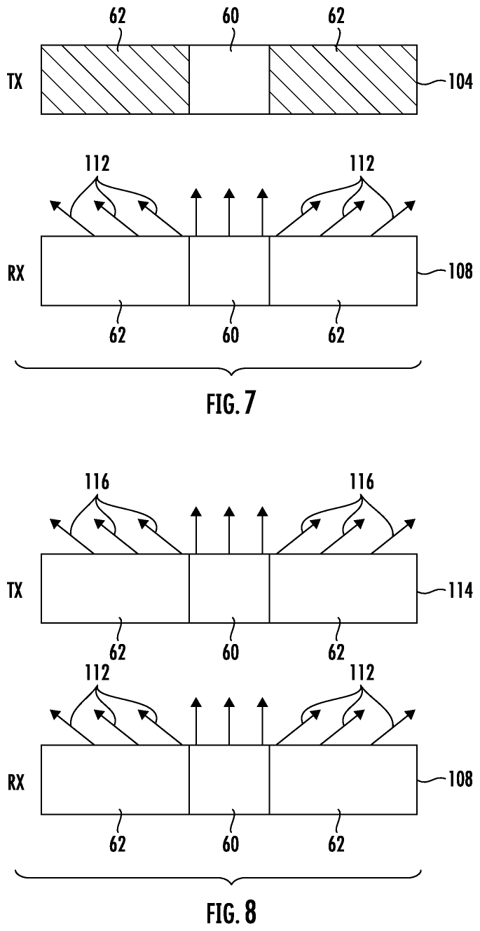

In the example shown in FIG. 8, the beam former of controller 36 applies an electrical bias pattern 114 during the transmit phase. With electrical bias pattern 114, a non-zero electrical bias is applied to the transducers 52 of aperture 60. In addition, a nonzero electrical bias is applied to at least one, a majority of, at least 90%, or all of the transducers 52 of portion 62. As discussed above, this nonzero electrical bias activates the transducers 52 of portion 62 to prevent parasitic capacitance. In the example illustrated, the non-zero electrical bias applied to the transducers 52 of portion 62 is timed or phased to steer or look away from aperture 60 as indicated by arrows 116. By steering (using bias voltages applied across the array elements) transducers 52 of portion 62 away from aperture 60, acoustic interference with the signals produced by the transducers 52 of aperture 60 is reduced or eliminated. With the method of FIG. 8, the strength of the signal across aperture 60 may be lower.

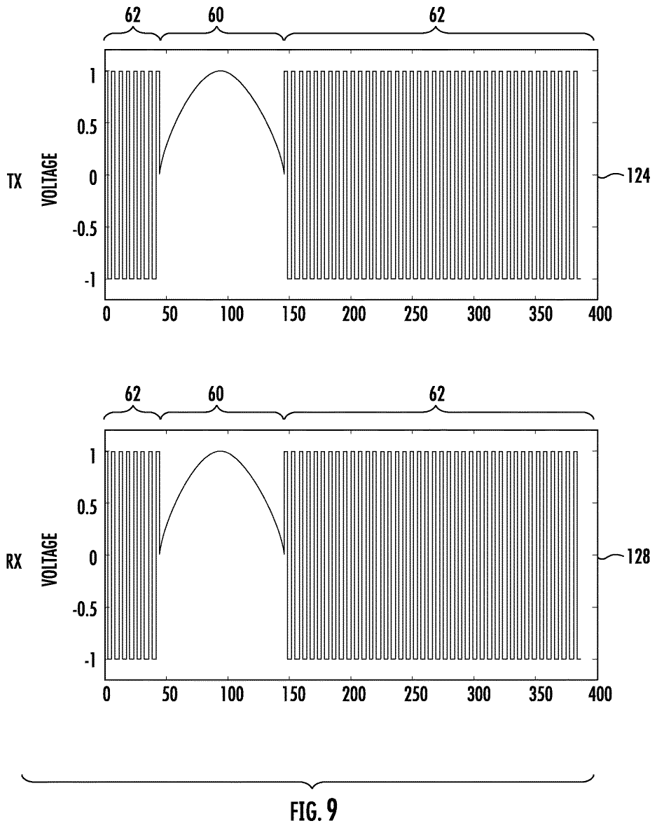

In the example shown in FIG. 9, the beam former of controller 36 applies a first electrical bias pattern 124 during the transmit phase and applies bias pattern 128 during the receive phase. Electrical bias patterns 124 and 128 both include the application of zero electrical bias to transducers 52 of both portion 60 and 62. Those transducers 52 of portion 62 to which are applied a non-zero electrical bias experience parasitic capacitance. To reduce or eliminate interference with the image signals from portion 60, patterns 124 and 128 both apply positive and negative electrical biases, such as +1 and −1, to transducers 52 and portion 62. The opposite polarity of the electrical bias results in cancellation of the signals from portion 62.

In some implementations, pattern 124 of FIG. 9 may be replaced with pattern 104 or pattern 114 described above. In some implementations, pattern 124 and pattern 128 may be configured such that the transducers 52 of portion 62 are additionally steered (using bias patterns) away from aperture 60 further reduce interference with the signals from the transducers 52 of aperture 60. As should be appreciated, patterns 124 and 128 may have other values than that are shown in FIG. 9.

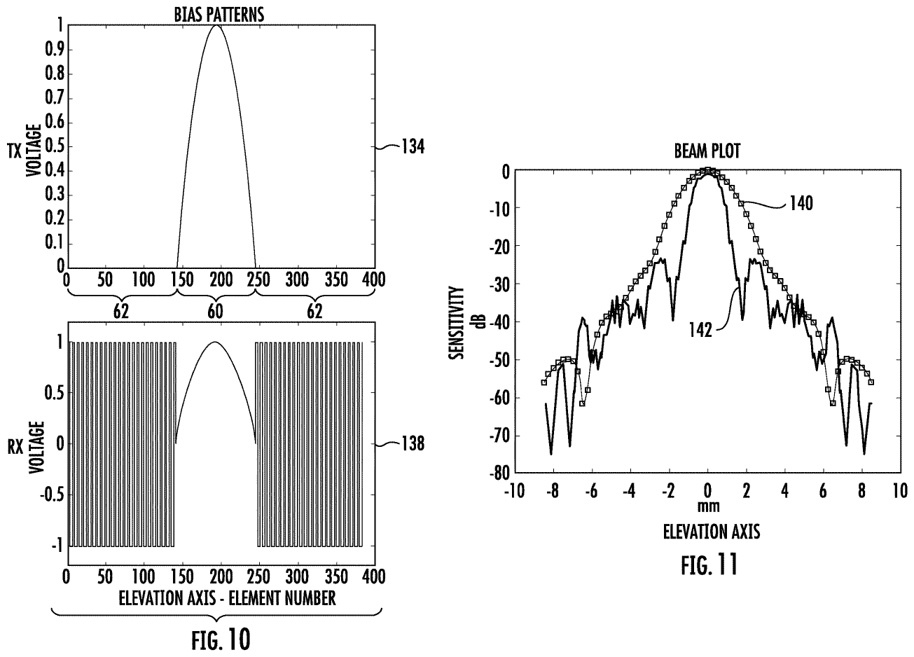

FIG. 10 illustrates an example of where the beam former controller 36 applies the first electrical bias pattern 134 to array 50 during the transmit phase and applies a second electrical bias pattern 138 to array 50 during the receive phase. Pattern 134 is similar to pattern 104 described above, wherein a nonzero electrical bias is applied to aperture 60 and a zero electrical bias is applied to portions 62 during the transmit phase. As discussed above, the strength of the signal may be sufficiently strong to overcome any parasitic capacitance.

Pattern 138 is similar to pattern 128 described above. Pattern 138 includes the application of both positive and negative electrical biases, such as +1 and −1, to transducers 52 in portion 62. The opposite polarity of the electrical bias results in cancellation of the signals from those transducers 52 of portion 62. FIG. 11 illustrates the example beam plot resulting from the element data produced by the application of patterns 134 and 138. The curve 140 with squares represents the beam plot for a single image. Each box in the plot is for a new scatterer x-position. The curve 142 is a beam plot resulting from the additional application of a synthetic aperture. Synthetic aperture processing consists of combining data collected with a moving sensor or array of sensors to generate a 2D or 3D image. The sensors could be moved mechanically (translations, rotations) or electronically (by selectively activating portions of a larger array).

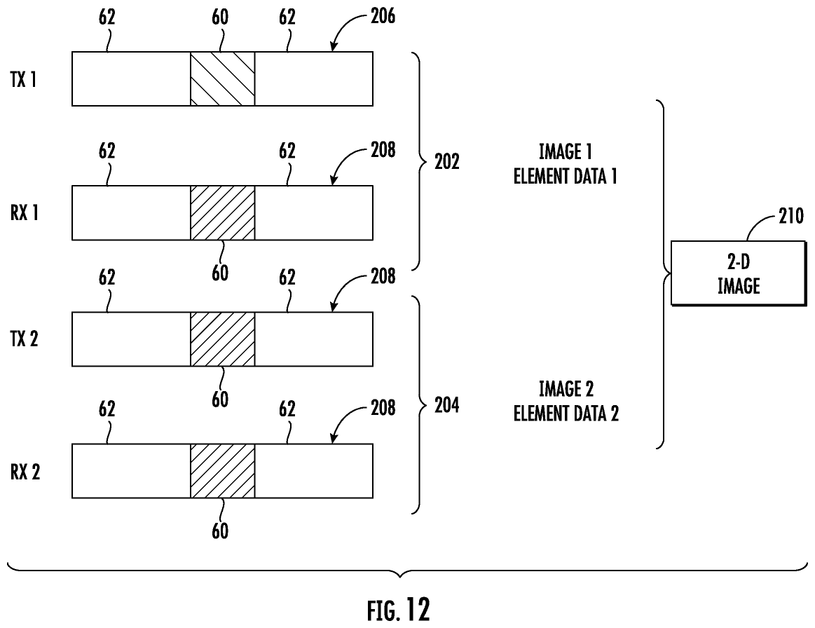

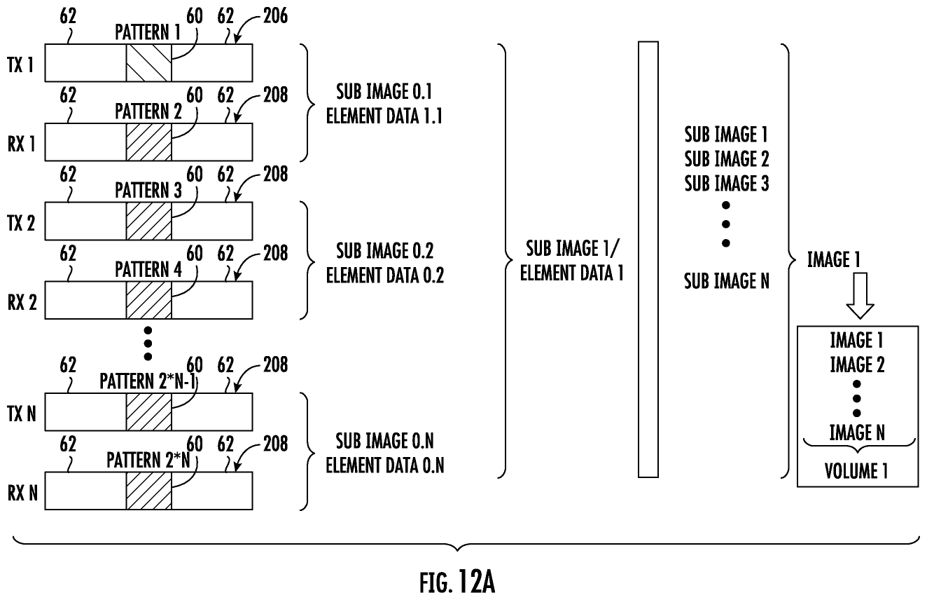

FIG. 12 illustrates another example of how the effects of activating transducers 52 in portion 62 with a non-zero bias may be mitigated. With the scheme shown in FIG. 12, the beam former of controller 36 carries out multiple transmit and receive phases for each particular position of aperture 60 along array 50. The electrical bias patterns applied to the first used portion of array 50, aperture 60, and the second unused portion 62 of array 50 are chosen such that the element data from such transmit/receive phases may be compared are combined to cancel out or eliminate the signal contributions from portion 62 so as to form the final 2D image for the particular positioning of aperture 60, wherein the final 2D image is to be used for forming the volume image.

This cancellation may occur at any one of various stages during processing of the element data. In some implementations, the raw/refined element data from each of the transmit/operations may be combined prior to forming an associated image, wherein during such combining, the cancellation of signals from portion 62 is performed. Thereafter, resulting element data may be used to form the final two-dimensional image.

In some implementations, the raw/refined element data from the multiple transmit/receive operations is first used to form intermediate two-dimensional images. The intermediate two-dimensional images are then combined to cancel the signal contributions of portion 62 and to form the final two-dimensional image.

In the example shown in FIG. 12, the beam former of controller 36 performs two transmit/receive operations 202, 204, wherein the element data from each of such operations 202, 204 is used directly or indirectly (as described above) to cancel out signal contributions from those transducers 52 of portion 62. During the first transmit/receive operation 202, the beam former controller 36 applies a first electrical bias pattern 206 during the transmit phase and a second electrical bias pattern 208 during the receive phase. Electrical bias pattern 206 includes the application of an electrical bias that substantially reduces or eliminates the radiation of acoustic energy or contribution of signal during receive from those transducers 52 of portion 60 (such as a zero bias or a small electrical bias) and the application of a non-zero bias to those transducers 52 of portion 62. Electrical bias pattern 208 includes the application of a non-zero bias to the transducers 52 of aperture 60 and the application of a non-zero bias to the transducers 52 of portion 62.

During the second transmit/receive operation 204, the beam former controller 36 applies electrical bias pattern 208 to array 50 during both the transmit and receive phases. The combination of the element data from operations 202, 204 or the combination of the intermediate images formed by or based upon the raw/refined element data of operations 202, 204 may be used to identify the particular signal contribution from those transducers 52 of aperture 60 and to identify the signal contributions from those transducers 52 of portion 62 for cancellation or removal. In some implementations, the electrical bias patterns 206 and/or 208 may additionally include the steering (using bias voltages applied across the array elements) of the transducers 52 of portion 62 away aperture 60 as described above with respect to FIGS. 7 and 8 and/or may additionally include the application of opposite polarity bias voltages to portion 62 for additional cancellation as discussed above with respect to FIG. 9.

Unless explicitly set forth, the term “image” may refer to a “whole image” or a partial or sub image. In some implementations, each set of transmit and receive bias pattern sequences or “operations” form a portion of a whole image. Using multiple partial or sub images to form a larger whole 2D image may occur where multiple focal points, plane waves, diverting waves and/or transmit/receive operations are used to form a single two-dimensional image. FIG. 12 illustrates an example where the raw/refined element data or the intermediate images correspond to whole two-dimensional images 210 of a region of interest. FIG. 12A illustrates an example ultrasound imaging method where multiple partial or sub images from multiple transmit/receive operations are used to form a whole two-dimensional image, wherein multiple whole two-dimensional images are then used to form a whole three-dimensional or volumetric image.

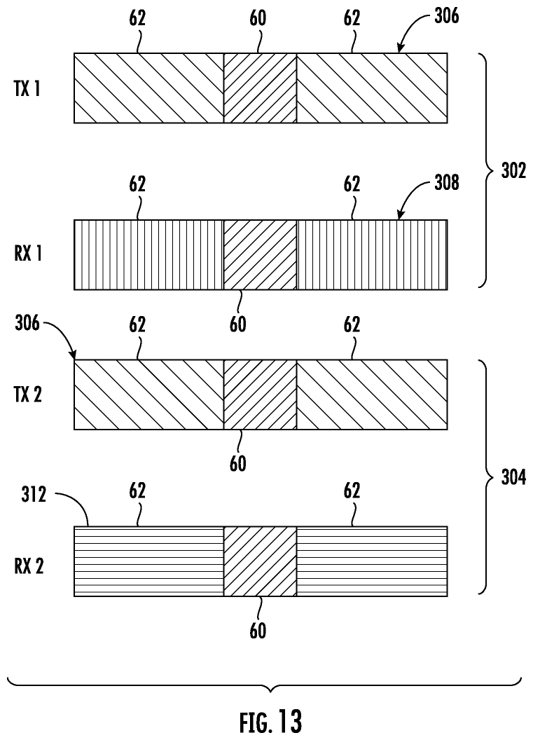

FIG. 13 schematically illustrates another example of how the electrical bias patterns applied to array 50 are applied during multiple transmit/receive operations may be used to cancel or remove signal contributions from those transducers 52 of the unused portion 62 of array 50. During operation 302, the beam former of controller 36 applies a first electrical bias pattern 306 during the transmit phase and a second electrical bias pattern 308 during the receive phase. Pattern 306 is similar to pattern 104 discussed above and includes the application of a nonzero bias to those transducers 52 of aperture 60 and the application of a nonzero bias to those transducers 52 of portion 62 during the transmit phase. Pattern 308 includes the application of a nonzero electrical bias to those transducers 52 of aperture 60 and the application of a nonzero electrical bias of those transducers 52 of portion 62 during the receive phase. As schematically shown, the nonzero electrical bias applied to transducers 52 of portion 62 have a first directionality (schematically illustrated as being vertical).

During operation 304, the beam former of controller 36 applies the electrical bias pattern 306 during the transmit phase and applies an electrical bias pattern 312 during the receive phase. Electrical bias pattern 312 includes the application of a nonzero electrical bias to those transducers 52 of aperture 60 and includes a nonzero electrical bias to those transducers 52 of portion 62. In contrast to pattern 308, pattern 312 applies a nonzero electrical bias to portion 62 that is orthogonal to the nonzero electrical bias applied to portion 62 with pattern 308. As result, the combination of the element data from the receiving phases of operations 302 and 304 results in the cancellation of the signal contributions from transducers 52 of portion 62 during the receiving phase.

In some implementations, the electrical bias patterns 306, 308 and/or 312 may additionally include the steering (using bias voltages) of the transducers 52 of portion 62 away aperture 60 as described above with respect to FIGS. 7 and 8 and/or may additionally include the application of opposite polarity bias voltages to portion 62 for additional cancellation as discussed above with respect to FIG. 9.

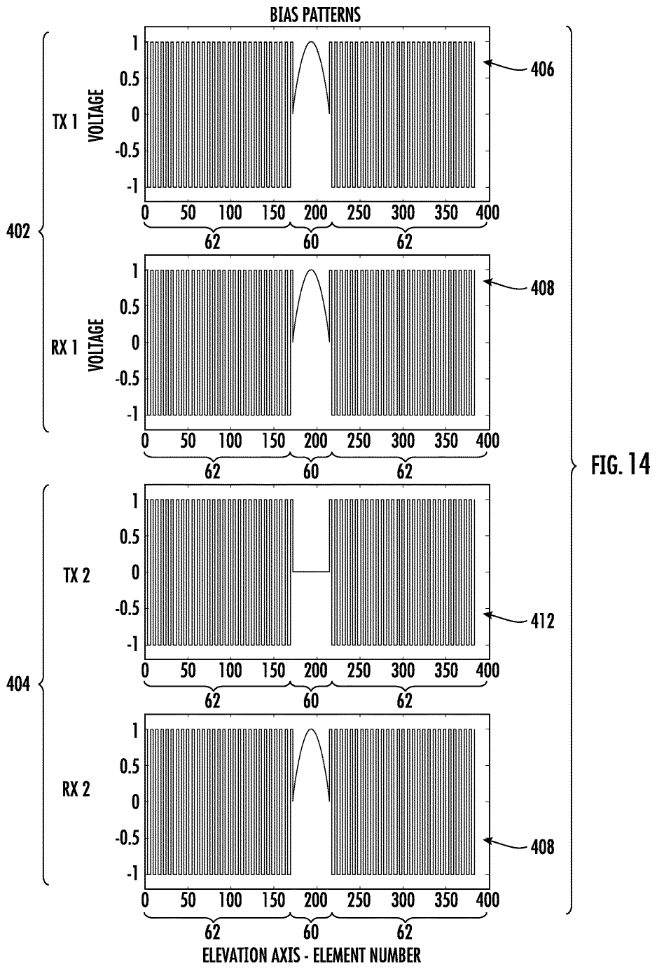

FIG. 14 illustrates another example of how the electrical bias patterns array 50 applied during multiple transmit/receive operations may be used to cancel or remove signal contributions from those transducers 52 of the unused portion 62 of array 50. During operation 402, the beam former of controller 36 applies a first electrical bias pattern 406 to array 50 during the transmit phase and a second electrical bias pattern 408 to array 50 during the receive phase. Patterns 406 and 408 are similar to patterns 124 and 128, respectively, described above. Patterns 406 and 408 each include the application of a nonzero bias to those transducers 52 of aperture 60 and the application of a nonzero bias to those transducers 52 of portion 62 during the transmit phase. Patterns 406 and 408 each include the application of a nonzero electrical bias to those transducers 52 of aperture 60 and the application of a nonzero electrical bias of those transducers 52 of portion 62 during the receive phase.

During operation 404, the beam former of controller 36 applies the electrical bias pattern 412 during the transmit phase and applies electrical bias pattern 408 during the receive phase. Electrical bias pattern 412 includes the application of a zero electrical bias (or a nonzero bias having a sufficiently small value to substantially reduce or eliminate the radiation of acoustic energy or contribution of signal) during receive to those transducers 52 of aperture 60 and includes a nonzero electrical bias to those transducers 52 of portion 62. As result, the combination of the element data from the transmit phases of operations 302 and 304 results in the cancellation of the signal contributions from transducers 52 of portion 62 during the transmit phase. In the example illustrated, the opposite polarities of the electrical bias voltage applied to transducers 52 of portion 62 during the receive phase mitigates or cancels signal contributions from portion 62 during the receive phase.

In some implementations, the electrical bias patterns 406, 408 and/or 412 may additionally include the steering (using bias voltages applied across the array elements) of the transducers 52 of portion 62 away aperture 60 as described above with respect to FIGS. 7 and 8. In some implementations, electrical bias patterns 408 may be replaced with electrical bias patterns similar to electrical bias patterns 308 and 312 described above with respect to FIG. 13.

FIG. 15 illustrates the example beam plot resulting from the element data produced by the application of operations 402, 404 and electrical bias patterns 406, 408 and 412. The curve 440 with squares represents the beam plot for a single image. The curve 442 is a beam plot resulting from the additional application of a synthetic aperture.

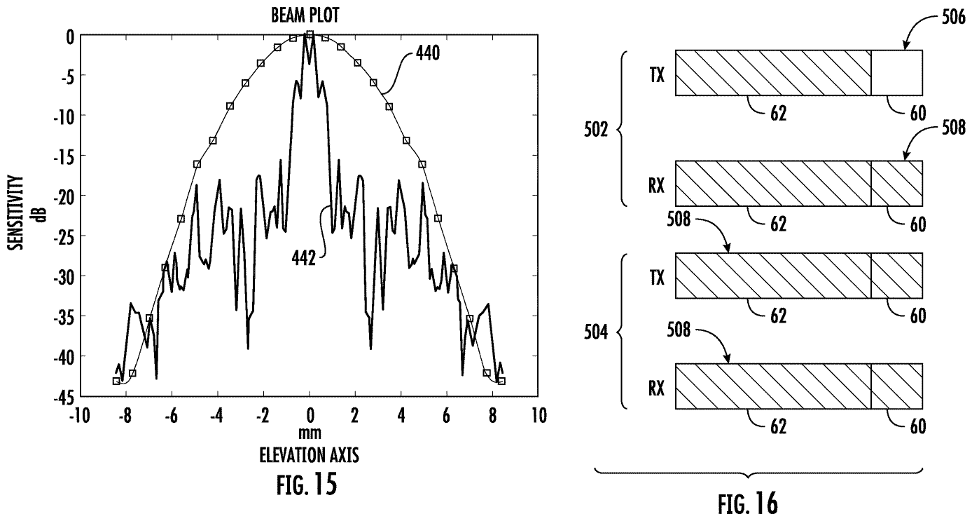

FIGS. 16-18 illustrate other example electrical bias pattern schemes that may be applied by the beam former of controller 36 to reduce or eliminate parasitic capacitance and to also reduce or eliminate acoustic signal contributions from the portion 62 of the array 50 that is not being used for a particular two-dimensional image. FIG. 16 includes transmit/receive operations 502, 504 which are combined or used in conjunction with one another to identify and cancel out the signal contributions from portion 62 for a particular two-dimensional image. As part of operation 502, the beam former of controller 36 applies the first electrical bias pattern 506 during a transmit phase and a second electrical bias pattern 508 during the receive phase. Electrical bias pattern 506 includes the application of a bias that substantially reduces or eliminates the radiation of acoustic energy or contribution of signal during receive from those transducers 52 of portion 60 and the application of a non-zero bias to those transducers 52 of portion 62. Electrical bias pattern 508 includes the application of a non-zero bias to the transducers 52 of aperture 60 and the application of a non-zero bias to the transducers 52 of portion 62.

During the second transmit/receive operation 504, the beam former of controller 36 applies electrical bias pattern 508 to array 50 during both the transmit and receive phases. The combination of the element data from operations 502, 504 or the combination of the intermediate images formed by or based upon the raw/refined element data of operations 502, 504 may be used to identify the particular signal contribution from those transducers 52 of aperture 60 and to identify the signal contributions from those transducers 52 of portion 62 for cancellation or removal. In some implementations, the electrical bias patterns 506 and/or 508 may additionally include the steering (using bias voltages applied across the array elements) of the transducers 52 of portion 62 away aperture 60 as described above with respect to FIGS. 7 and 8 and/or may additionally include the application of opposite polarity bias voltages to portion 62 for additional cancellation as discussed above with respect to FIG. 9.

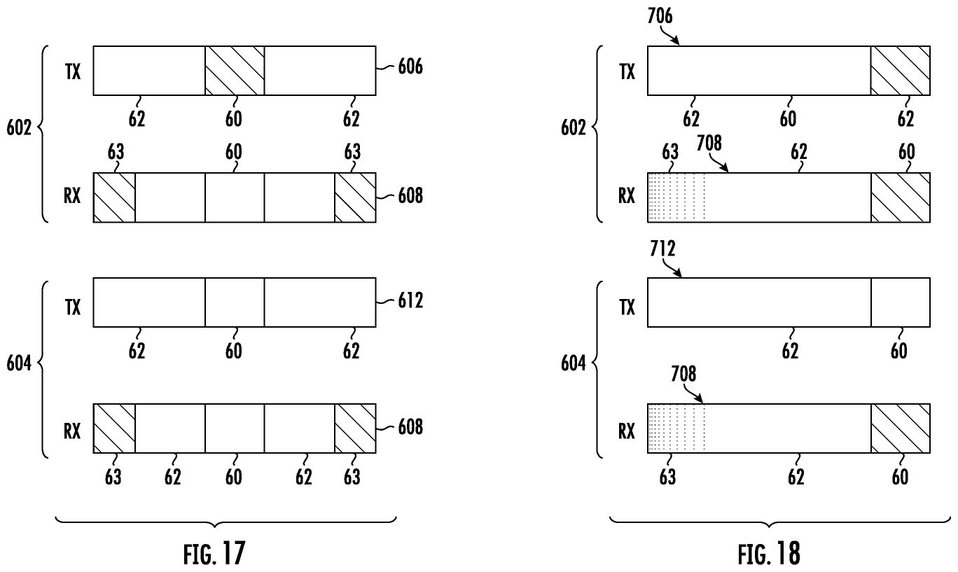

FIG. 17 illustrates an electrical bias pattern scheme applied by the beam former of controller 36, the scheme including operations 602 and 604. The scheme shown in FIG. 17 illustrates an example of where the first portion forming aperture 60 and the second unused portion 62 do not encompass the entirety of array 50. As part of operation 602, the beam former of controller 36 applies the first electrical bias pattern 606 during a transmit phase and a second electrical bias pattern 608 during the receive phase. Electrical bias pattern 606 includes the application of a bias that substantially reduces or eliminates the radiation of acoustic energy or contribution of signal during receive from those transducers 52 of portion 60 (such as a zero bias or an otherwise sufficiently small non-zero bias) and the application of a non-zero bias (that is sufficient to reduce or eliminate parasitic capacitance) to those transducers 52 of portion 62. Electrical bias pattern 608 includes the application of a non-zero bias to the transducers 52 of aperture 60 and the application of a non-zero bias to the transducers 52 of portion 62. Electrical bias pattern 608 additionally includes the application of bias that substantially reduces or eliminates the radiation of acoustic energy or contribution of signal during receive (such as a zero bias or otherwise sufficiently small non-zero bias) from portion 63. For example, portion 63 comprises portions of array 50 most distant from aperture 60.

As part of operation 604, the beam former of controller 36 applies electrical bias pattern 612 during the transmit phase and applies electrical bias pattern 608 during the receive phase. Electrical bias pattern 612 includes the application of a non-zero electrical bias voltage to those transducers 52 of both portion 60 and 62. As with the approach shown in FIG. 16, the approach shown in FIG. 17 facilitates the cancellation or removal of signal contributions from those transducers 52 of portion 62 of array 50.

FIG. 18 illustrates an example electrical bias pattern scheme applied by the beam former of controller 36, the scheme including operations 702 and 704. The scheme shown in FIG. 18 illustrates an example of how the electrical bias voltages applied across the unused portion 62 of array 50 may taper or gradually vary across portion 62. As part of operation 702, the beam former of controller 36 applies the first electrical bias pattern 706 during a transmit phase and a second electrical bias pattern 708 during the receive phase. Electrical bias pattern 706 includes the application of a bias that substantially reduces or eliminates the radiation of acoustic energy or contribution of signal during receive (such as a zero bias or otherwise sufficiently small non-zero bias) from those transducers 52 of portion 60 (resulting in the transducers 52 of aperture 60 being in an inactive state) and the application of a non-zero bias to those transducers 52 of portion 62 (resulting in the transducers 52 of portion 62 being in an active state). Electrical bias pattern 708 includes the application of a non-zero bias to the transducers 52 of aperture 60 and the application of a nonuniform distribution of non-zero biases to the transducers 52 of portion 62. In the example illustrated, the nonuniform distribution is gradual or tapers based upon the distance of aperture 60. In other implementations, the nonuniform distribution of nonzero biases to the transducers 52 of portion 62 may vary in other manners. Electrical bias pattern 708 additionally includes the application of a bias that substantially reduces or eliminates the radiation of acoustic energy or contribution of signal (such as a zero bias or otherwise sufficiently small non-zero bias) during receive from portion 63. For example, portion 63 comprises portions of array 50 most distant from aperture 60.

As part of operation 604, the beam former of controller 36 applies electrical bias pattern 712 during the transmit phase and applies electrical bias pattern 708 during the receive phase. Electrical bias pattern 712 includes the application of a non-zero electrical bias voltage to those transducers 52 of both portions 60 and 62. As with the approaches shown in FIGS. 16 and 17, the approach shown in FIG. 18 facilitates the cancellation or removal of signal contributions from those transducers 52 of portion 62 of array 50.

FIGS. 1-18 illustrate various examples of schemes, approaches or manners by which different electrical bias patterns may be applied across the array 50 when gathering element data performing a particular two-dimensional image correspond to a particular position of the aperture 60 being walked across the array 50. It should be appreciated that the non-zero electrical bias voltages being applied in the above examples may vary across portion 62 and such or across aperture 60. It should be appreciated that any of the above methods or techniques for reducing or eliminating signal contributions from portion 62 may be used independent of one another or may be combined with one another. It should be appreciated that the size of aperture 60 during the transmit phase and the receiving phase of a transmit/receive operation may vary. For example, the size of aperture 60 during the transmit phase may be different than the size of the aperture 60 during the receive phase. Similarly, the size of aperture 60 during a first data element gathering operation may be different from the size of aperture 60 during a second data element gathering operation of a particular scheme or approach.

Likewise, the size of the unused portion 62 may vary. For example, the size of portion 62 during the transmit phase may be different than the size of the portion 62 during the receive phase. Similarly, the size of portion 62 during a first data element gathering operation may be different from the size of portion 62 during a second data element gathering operation of a particular scheme or approach. As discussed above, portion 62 and aperture 60 may not collectively extend across the entirety of array 50. In some implementations, some of the transducers 52 in an active aperture 60 or in portion 62 may remain inactive (having an applied nonzero bias or a non-zero bias having a value that substantially reduces or eliminates the radiation of acoustic energy or contribution of signal during receive.

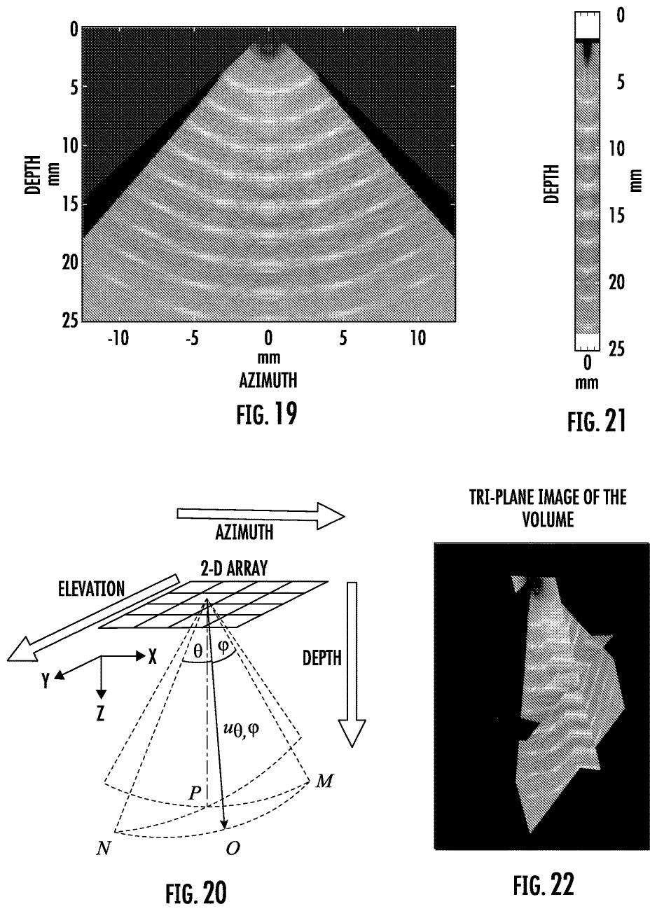

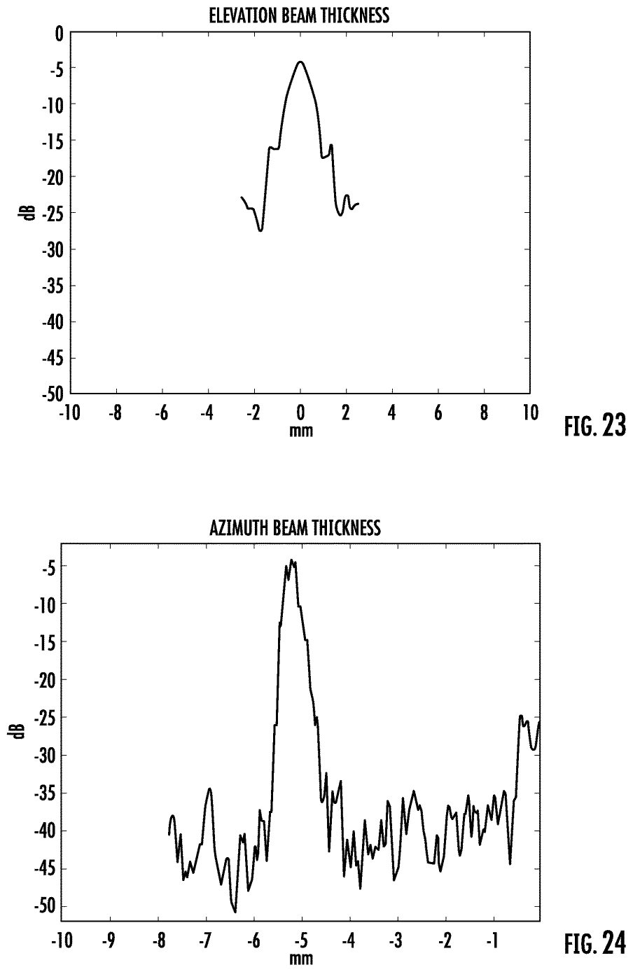

FIGS. 19-24 illustrate an example simulation in which the beam former of controller 36 applies the electrical bias pattern scheme discussed above with respect to FIGS. 14 and 15. For example, FIG. 19 depicts a slice of a volume image. FIG. 21 illustrates an orthogonal image slice (into the view of FIG. 19). FIG. 22 depicts a tri-plane image of the volume. FIGS. 23 and 24 describe the beam thickness in the elevation and azimuth directions.

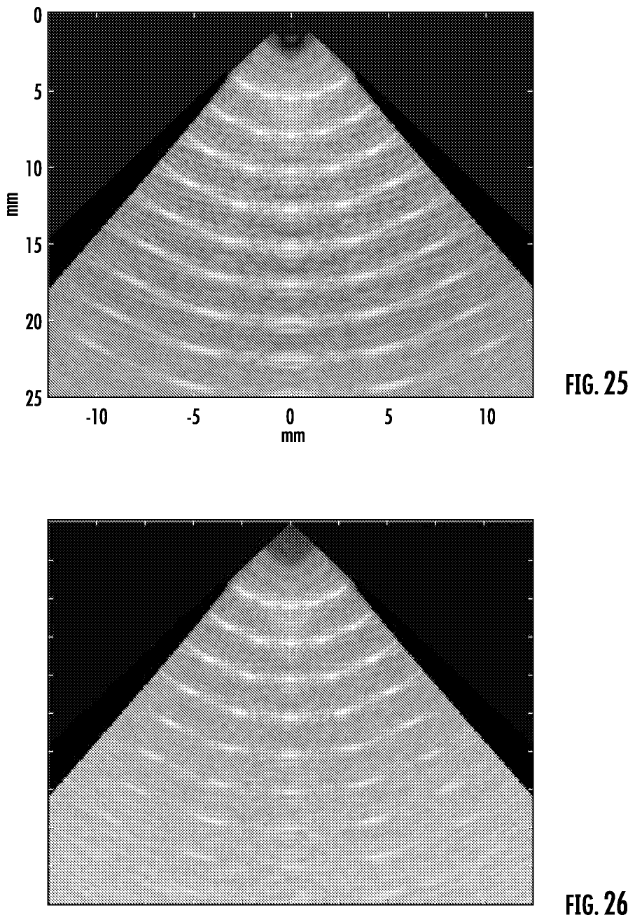

FIGS. 25 and 26 comprise simulated ultrasound imaging examples. FIG. 25 illustrates a slice of a volume generated pursuant to the method 64 described above with respect to FIG. 5 wherein a zero bias is applied to unused portions of array 50 when capturing element data for each of the two-dimensional images that form the volume image. FIG. 26 illustrates a slice of the same volume generated pursuant to the method 74 described above with respect to FIG. 5, wherein a smart bias or a non-zero bias is applied to those unused portions of array 50 when capturing element data for each of the two-dimensional images that form the volume image.

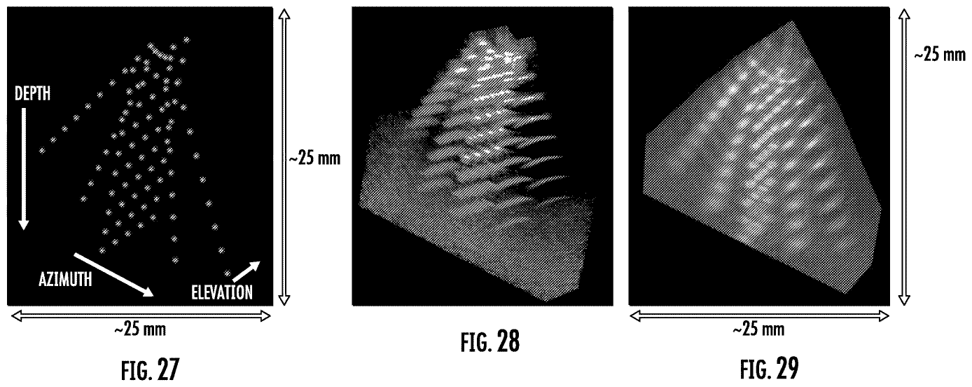

FIGS. 27-29 illustrate an example simulation of the application of synthetic aperture processing. FIG. 27 illustrates various point targets. FIG. 28 illustrates an example volume image formed using method 74 without the application of synthetic aperture processing. FIG. 29 illustrates an example volume image formed using method 74 with synthetic aperture processing. In addition to the synthetic aperture processing, the image processor of controller 36 may optionally apply quadrature processing. Quadrature processing combines the data from 4 transmissions with Fresnel lens apodization on the bias lines to achieve an elevational focus comparable in quality to delay based focusing methods.

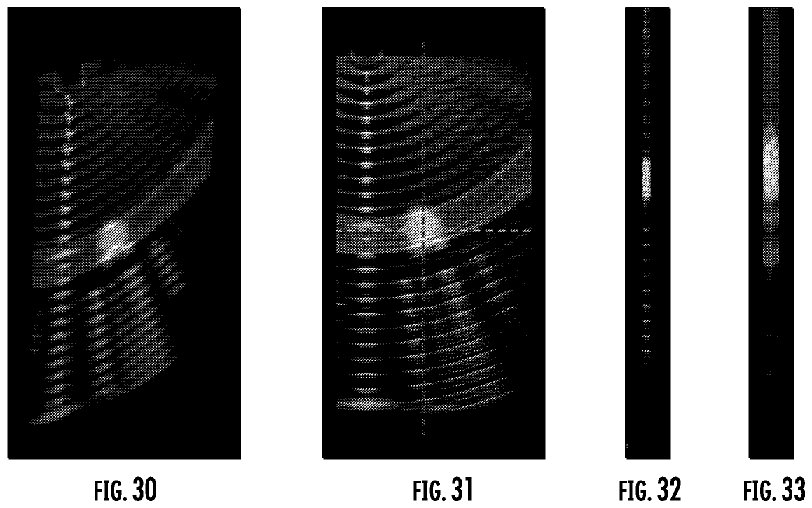

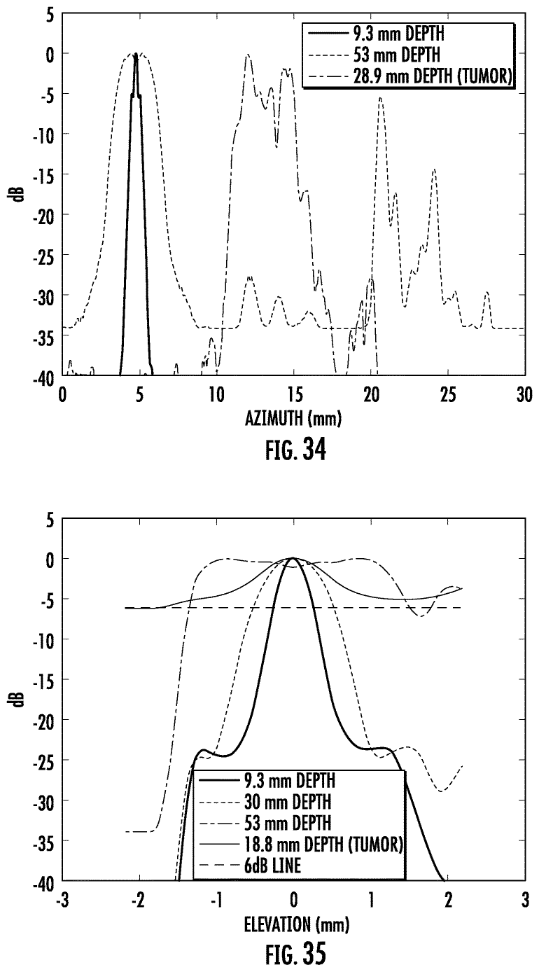

FIGS. 30-33 are simulations of ultrasound images illustrating a slice of a volume generated pursuant to method 64 described above respect to FIG. 5, wherein a zero bias is applied to unused portions of array 50 and capturing element data for each of the two-dimensional images that form the volumetric image. FIG. 30 is a maximum projection rendering of the volume which shows point targets and spherical hyperechoic target. FIG. 31 is a 2D image slice through the elevation axis from the volume showing point targets extending and the hyperechoic target. FIG. 32 is a 2D image slice through the azimuthal axis showing the point targets and hyperechoic target. FIG. 33 is a 2D image slice through the depth axis from the volume showing the hyperechoic target. FIGS. 34 and 35 are beam plots of the volumetric image shown in FIGS. 30-33 in the azimuth and elevation directions. The beam plots depict a resolution of 0.56 mm at a depth of and a resolution of 2.6 mm at a depth of 53 mm. The simulations do not reflect the impact of parasitic capacitance which might otherwise impact contrast and penetration depth.

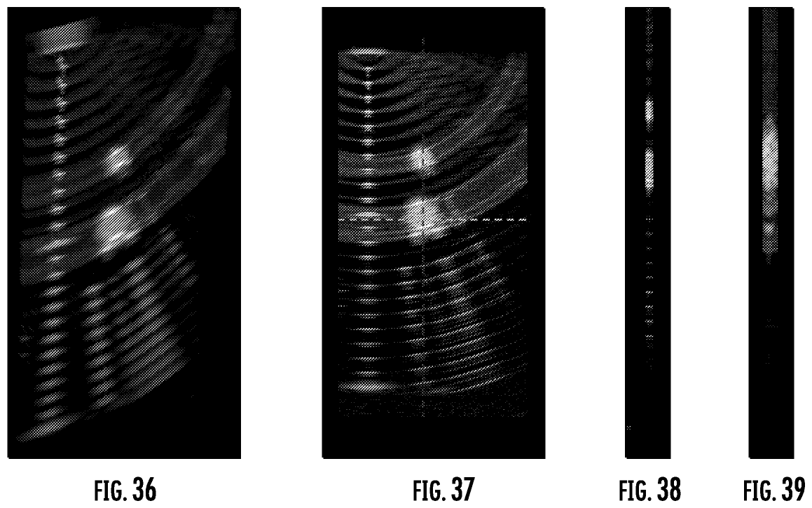

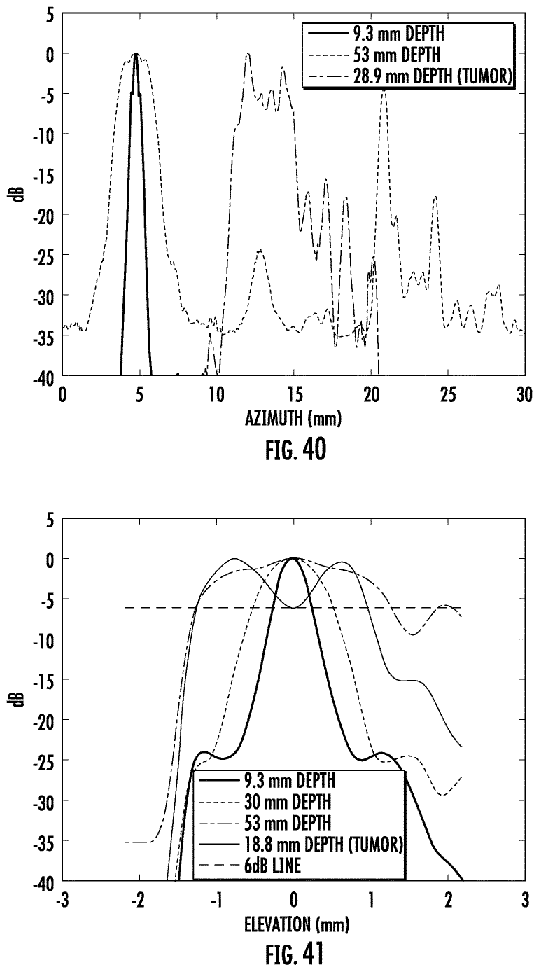

For purposes of comparison, FIG. 36-39 are simulations of ultrasound images of the exact same environment imaged in FIGS. 30-33 illustrating a slice of a volume generated pursuant to method 74 described above respect to FIG. 5, wherein a non-zero bias is applied to unused portions of array 50 when capturing element data for each of the two-dimensional images that form the volumetric image. FIG. 36 is a maximum projection rendering of the volume which shows point targets and two spherical hyperechoic targets. FIG. 37 is a 2D image slice through the elevation axis from the volume showing point targets extending and the hyperechoic targets. FIG. 38 is a 2D image slice through the azimuthal axis showing the point targets and hyperechoic targets. FIG. 39 is a 2D image slice through the depth axis from the volume showing the hyperechoic target. FIGS. 40 and 41 are beam plots of the volumetric image shown in FIGS. 30-33 in the azimuth and elevation directions. The beam plots depict a resolution of 0.56 mm at a depth of and a resolution of 2.6 mm at a depth of 53 mm. As shown by FIGS. 36-41, the images produced by method 74 are equivalent to the images produced by method 64 absent parasitic capacitance which results in image degradation. The application of the non-nonzero electrical bias to those unused portions of the array 50 during the formation of each two-dimensional image does not degrade the two-dimensional images and the resulting volumetric image and eliminates or reduces parasitic capacitance to enhance penetration depth and image contrast.

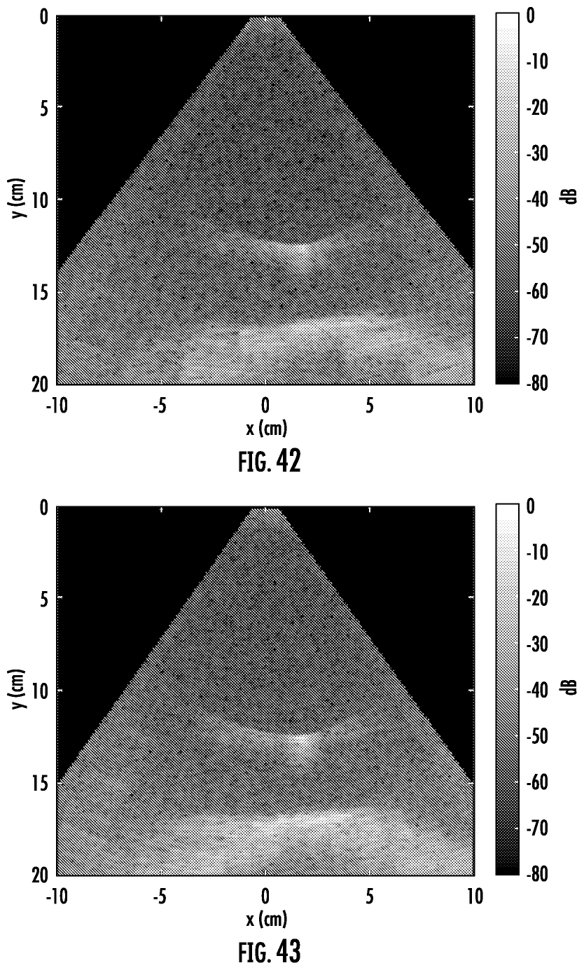

For purposes of comparison, FIGS. 42 and 43 illustrate slices of volumetric or volume images output by an ultrasound imaging system. FIGS. 42 and 43 are not the result of a simulation. Each of such images is that of a wire target in water. Each of the two-dimensional images, which are combined to form the volume image, is generated on a 128 signal line×64 bias line array where 12 bias lines are used to form and walk the aperture.

FIG. 42 illustrates a slice of a volume generated by hardware pursuant to method 54 described above with respect to FIG. 5, wherein a nonzero bias is applied to unused portions of array 50 when capturing element data for each of the two-dimensional images that form the volume image. FIG. 43 is an ultrasound image of the exact same environment (wire target in water) imaged in FIG. 42 illustrating a slice of a volume generated pursuant to method 74 described above respect to FIG. 5, wherein a non-zero bias is applied to unused portions of array 50 when capturing element data for each of the two-dimensional images that form the image. As revealed by the comparison of FIGS. 42 and 43, the application of the non-nonzero bias to unused portions of the array 50 when capturing element data for each of the two-dimensional images that form the volume image does not result in degradation of image quality or penetration depth.

Although the claims of the present disclosure are generally directed to an ultrasound imaging system that generates a volumetric image based on different two-dimensional images that are based on signals from different first portions of an array of ultrasonic transducers, wherein a non-zero electrical bias is applied to second unused portions of the array during the receiving phase to reduce parasitic capacitance of the unused portions, the present disclosure is additionally directed to the features set forth in the following definitions.
1. An ultrasound imaging system comprising:
an array of ultrasonic transducers; and
a controller configured to:
selectively activate different portions of the array to determine whole or partial different two-dimensional images, each of the whole or partial two-dimensional images being based upon signals that are estimated to be received from a different first portion of the array,
   wherein each of the whole or partial two-dimensional images is based on element data output by the ultrasonic transducers of an array where a first electrical bias pattern is applied to a first and second portion of the array during a transmitting phase and a second electrical bias pattern is applied to the first portion and the second portion of the array during the receiving phase, wherein the second electrical bias
      pattern includes a non-zero electrical bias applied to at least one ultrasonic transducer of the second portion; and
   generate a volumetric image based on the different whole or partial two-dimensional images.
2. The system of definition 1, wherein the first electrical bias pattern includes a non-zero electrical bias applied to at least one ultrasonic transducer of the second portion.
3. The system of definition 1, wherein the first electrical bias pattern comprises positive and negative electrical biases for the second portion to cancel sensing by the second portion.
4. The system of definition 1, wherein the second electrical bias pattern comprises positive and negative electrical biases for the second portion to cancel sensing by the second portion.
5. The system of definition 1, wherein the ultrasonic transducers of the second portion are steered away from the first portion during at least one of the transmission phase and the receiving phase.
6. The system of definition 1, wherein the second electrical bias pattern comprises a tapered bias pattern.
7. The system of definition 1, wherein each of the two-dimensional images is additionally based on a combination of the element data and second element data, the second element data being output by the ultrasonic transducers of the first portion of the array in response to application of a third electrical bias pattern applied to the first portion and a second portion of the array during a second transmitting phase and a fourth electrical bias pattern applied to the first portion and the second portion of the array during a second receiving phase,
wherein the third electrical bias pattern and the fourth electrical bias pattern each include a non-zero electrical bias applied to at least one ultrasonic transducer of the second portion.
8. The system of definition 7, wherein the first electrical bias patten does not apply an appreciable electrical bias to the first portion and wherein the third electrical bias pattern applies an electrical bias to at least one ultrasonic transducer of the first portion.
9. The system of definition 7, The system of claim 1, wherein at least one of the third electrical bias pattern and the fourth electrical bias pattern comprises positive and negative electrical biases for the second portion to cancel sensing by the second portion.
10. The system of definition 7, wherein the ultrasonic transducers of the second portion are steered away from the first portion during at least one of the second transmission phase and the second receiving phase.
11. The system of any of definitions 7-10, wherein combination comprises a comparison selected from a group of comparisons consisting of: (1) a subtraction operation on the first set of element data and the second set of element data to form a particular two-dimensional image; (2) a subtraction operation on a first image based on the first and second sets of element data, and a second image based on the third and fourth sets of element data; (3) a subtraction operation on a first image based on the first and second sets of element data, and a second image based on the third and fourth sets of element data following scan conversion of the first and second images; (4) a subtraction operation on a first image based on the first and second sets of element data, and a second image based on the third and fourth sets of element data following scan conversion of the first and second images and following compression of the first and second images.
12. The system of definition 1, wherein at least one of the third electrical bias pattern and the fourth electrical bias pattern comprises a tapered bias pattern.
13. The system of definition 7, wherein the second electrical bias pattern, the third electrical bias pattern and the fourth electrical bias pattern are similar.
14. The system of any of definitions 1-13, wherein the array of ultrasonic transducers comprises a third portion of ultrasonic transducers.
15. The system of any of definitions 1-14, wherein the first portion of the array during the transmission phase and the first portion of the array during the receiving phase are dissimilar.
16. The system of any of definitions 1-15, wherein the second portion of the array during the transmission phase and the second portion of the array during the receiving phase are dissimilar.
17. The system of any of definitions 1-16, wherein the array of ultrasonic transducers is a type selected from a group of types consisting of: an electrostrictive transducer array; a capacitive micromachined ultrasound transducer (CMUT) array; and a piezoelectric micromachined ultrasound transducer array (PMUT).
18. The system of any of definitions 1-17, wherein each ultrasonic transducer comprises first and second conductive layers spaced by a dielectric layer therebetween.
19. The system of any of definitions 1-18, wherein the controller is further configured to apply synthetic aperture processing.
20. The system of any of definitions 1-19, wherein the different first portions of the array are used to determine the different two-dimensional images comprise overlapping subsets of the array.
21. The system of any of definitions 1-19, wherein the array is planar.
22. The system of any of definitions 1-20, wherein the array is curved.
23. The system of any of definitions 1-22, wherein the array is two-dimensional

Although the present disclosure has been described with reference to example implementations, workers skilled in the art will recognize that changes may be made in form and detail without departing from the scope of the claimed subject matter. For example, although different example implementations may have been described as including features providing benefits, it is contemplated that the described features may be interchanged with one another or alternatively be combined with one another in the described example implementations or in other alternative implementations. Because the technology of the present disclosure is relatively complex, not all changes in the technology are foreseeable. The present disclosure described with reference to the example implementations and set forth in the following claims is manifestly intended to be as broad as possible. For example, unless specifically otherwise noted, the claims reciting a single particular element also encompass a plurality of such particular elements. The terms “first,” “second,” “third” and so on in the claims merely distinguish different elements and, unless otherwise stated, are not to be specifically associated with a particular order or particular numbering of elements in the disclosure.

1. An ultrasound imaging system comprising:
an array of ultrasonic transducers; and
a controller configured to:
selectively activate different portions of the array to determine whole or partial different two-dimensional images, each of the whole or partial two-dimensional images being based upon signals that are estimated to be received from a different first portion of the array,
wherein each of the whole or partial two-dimensional images is based on element data output by the ultrasonic transducers of an array where a first electrical bias pattern is applied to a first and second portion of the array during a transmitting phase and a second electrical bias pattern is applied to the first portion and the second portion of the array during the receiving phase,
wherein the second electrical bias pattern includes a non-zero electrical bias applied to at least one ultrasonic transducer of the second portion; and
generate a volumetric image based on the different whole or partial two-dimensional images.

2. The system of claim 1, wherein the first electrical bias pattern includes a non-zero electrical bias applied to at least one ultrasonic transducer of the second portion.

3. The system of claim 1, wherein the first electrical bias pattern comprises positive and negative electrical biases for the second portion to cancel sensing by the second portion.

4. The system of claim 1, wherein the second electrical bias pattern comprises positive and negative electrical biases for the second portion to cancel sensing by the second portion.

5. The system of claim 1, wherein the ultrasonic transducers of the second portion are steered away from the first portion during at least one of the transmission phase and the receiving phase.

6. The system of claim 1, wherein the second electrical bias pattern comprises a tapered bias pattern.

7. The system of claim 1, wherein each of the two-dimensional images is additionally based on a combination of the element data and second element data, the second element data being output by the ultrasonic transducers of the first portion of the array in response to application of a third electrical bias pattern applied to the first portion and a second portion of the array during a second transmitting phase and a fourth electrical bias pattern applied to the first portion and the second portion of the array during a second receiving phase,
wherein the third electrical bias pattern and the fourth electrical bias pattern each include a non-zero electrical bias applied to at least one ultrasonic transducer of the second portion.

8. The system of claim 7, wherein the first electrical bias patten does not apply an appreciable electrical bias to the first portion and wherein the third electrical bias pattern applies an electrical bias to at least one ultrasonic transducer of the first portion.

9. The system of claim 7, wherein at least one of the third electrical bias pattern and the fourth electrical bias pattern comprises positive and negative electrical biases for the second portion to cancel sensing by the second portion.

10. The system of claim 7, wherein the ultrasonic transducers of the second portion are steered away from the first portion during at least one of the second transmission phase and the second receiving phase.

11. The system of claim 1, wherein combination comprises a comparison selected from a group of comparisons consisting of: (1) a subtraction operation on the first set of element data and the second set of element data to form a particular two-dimensional image; (2) a subtraction operation on a first image based on the first and second sets of element data, and a second image based on the third and fourth sets of element data; (3) a subtraction operation on a first image based on the first and second sets of element data, and a second image based on the third and fourth sets of element data following scan conversion of the first and second images; (4) a subtraction operation on a first image based on the first and second sets of element data, and a second image based on the third and fourth sets of element data following scan conversion of the first and second images and following compression of the first and second images.

12. The system of claim 1, wherein at least one of the third electrical bias pattern and the fourth electrical bias pattern comprises a tapered bias pattern.

13. The system of claim 7, wherein the second electrical bias pattern, the third electrical bias pattern and the fourth electrical bias pattern are similar.

14. The system of claim 1, wherein the array of ultrasonic transducers comprises a third portion of ultrasonic transducers.

15. The system of claim 1, wherein the different first portions of the array are used to determine the different two-dimensional images comprise overlapping subsets of the array.
